(19) United States
(12) Patent Application Publication
(10) Pub. No.: US 2007/0270629 A1
(43) Pub. Date: Nov. 22, 2007

(54) SYSTEM AND TECHNIQUES FOR MAGNETIC MANIPULATION OF INTERNAL ORGANS DURING MINIMALLY INVASIVE SURGERY

(76) Inventor: Filipi J. Charles, Omaha, NE (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/750,670

(22) Filed: May 18, 2007

Related U.S. Application Data
(60) Provisional application No. 60/802,207, filed on May 19, 2006.

(51) Int. Cl. A61N 2/00

(52) U.S. Cl. 600/12

(57) ABSTRACT

Systems and techniques for manipulating organs during minimally invasive surgery are disclosed. Magnetic material is delivered to an organ (10) of interest and the organ (10) is manipulated during the procedure via a magnet (30) external to the patient (25). The magnetic material may be delivered in fluidized form by filing a balloon (22).

### RELATED APPLICATION DATA

This application claims the benefit of U.S. Provisional Ser. No. 60/802,207 filed May 19, 2006.

### BACKGROUND

The present invention is related to the manipulation of internal organs during minimally invasive surgery. More particularly, but not exclusively, the present invention provides systems and techniques for surgically inserting magnetic material into or onto an organ such that the organ can be manipulated via an external magnet.

### SUMMARY

In one form, the present invention involves inserting magnetic material into an organ and then manipulating the organ during its removal with an external magnet. The manipulation is accomplished by utilizing the magnetic attraction or repulsion between the external magnet and the magnetic material inserted into the organ.

To insert the magnetic material into (or onto) the organ, an access path into (or adjacent) the organ is first established with a suitable delivery mechanism, such as a hollow needle or catheter, the working channel of an endoscope channel, or a similar device having at least one internal lumen. The magnetic material is then provided through the lumen of the delivery mechanism into the organ (or attached externally to the organ, or a combination thereof). With the organ thusly magnetized, the organ may be manipulated with the external magnetic device.

The external magnetic device is located outside the patient and may be attached to an articulating arm. The arm may be attached to the operating table or otherwise positioned in the operating room. Alternatively or in addition, the external magnet may be held in position (e.g. manually) by operating room personnel or affixed to the patient's body.

The magnetic material that is positioned on or in the organ can take various forms. In one form, the magnetic material is a number of beads that are injected into the organ. In another form, a flexible container, such as a balloon is inserted into the organ and then filled with a liquid magnetic material. In another form, a wire at least partially constructed of magnetic material is coiled inside the organ. In another form, an expandable structure having a magnetic component is inserted into and then expanded inside the organ. In still another form, a device having a magnetic component is attached to the organ, for example by being stapled, sutured, or clamped onto the organ.

The magnetic material placed into or onto the organ can be actively magnetic or passively magnetic. An actively magnetic device is a source of a magnetic field. Examples of actively magnetic devices are permanent magnets and electromagnets. A passively magnetic device is not itself a magnetic field source, but it is magnetically responsive to a magnetic field (e.g., it is magnetically attracted to an actively magnetic device). An example of a passively magnetic device is a body of initially unmagnetized ferromagnetic material. As used herein, the phrase “magnetic device” or “magnetic material” generally refers to both actively and passively magnetic devices/materials. However, it should be understood that in any system of multiple magnetic devices there will generally need to be at least one actively magnetic device, for example a passively magnetic material might be injected in the organ and manipulated by an actively magnetic device external to the body.

The present techniques can be applied to any type of surgery where an internal organ needs to be manipulated by a surgeon, for example where the organ it itself the object of the surgery (i.e. organ removal or repair) or where the organ needs to be moved to provide access to another organ that is the object of the surgery. It is expected to be of benefit during minimally invasive (e.g. endoscopic or laparoscopic) surgeries, with particular benefit in the context of minimally invasive surgeries conducted at least partially via a natural body orifice, for example those referred to as Natural Orifice Transluminal Endoscopic Surgery (NOTES).

In one type of natural orifice surgery, the surgeon insets a flexible endoscope through the patient's mouth and into the gastric cavity. A hole is established in the stomach wall, and the endoscope penetrates into the peritoneal cavity through the hole in the stomach wall. The hole may be kept open by balloon dilation. Using this access route, a surgeon can operate on various indications that would normally require open or minimally invasive access to the abdominal cavity via incisions in the skin. After the operation, the endoscope is withdrawn through the stomach wall and the incision in the stomach wall is closed. Reducing or eliminating the need for incisions in the skin reduces the discomfort and/or risk to the patient.

### BRIEF DESCRIPTION OF THE FIGURES

FIGS. 1-5 schematically depicts the delivery of magnetic material into a gall bladder by inserting a balloon into the gall bladder and then filling the balloon with magnetic fluid.

FIG. 6 schematically depicts the manipulation of the gall bladder containing the filled balloon with an external magnet.

FIG. 7 schematically illustrates an external magnet mounted to an articulating arm for use in manipulating an internal organ.

FIG. 8 schematically illustrates a self-expanding magnetic device being delivered into an organ via a needle injected through the skin.

FIG. 9 schematically illustrates the magnetic device of FIG. 8 in its expanded condition inside the organ.

FIG. 10 is a side view of a magnetic device adapted to be clamped onto an organ.

FIG. 11 is a side view of another magnetic device adapted to be clamped onto an organ.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is hereby intended. Alterations and further modifications in the illustrated devices, and such further applications of the principles of the invention as illustrated herein are contemplated as would normally occur to one skilled in the art to which the invention relates.

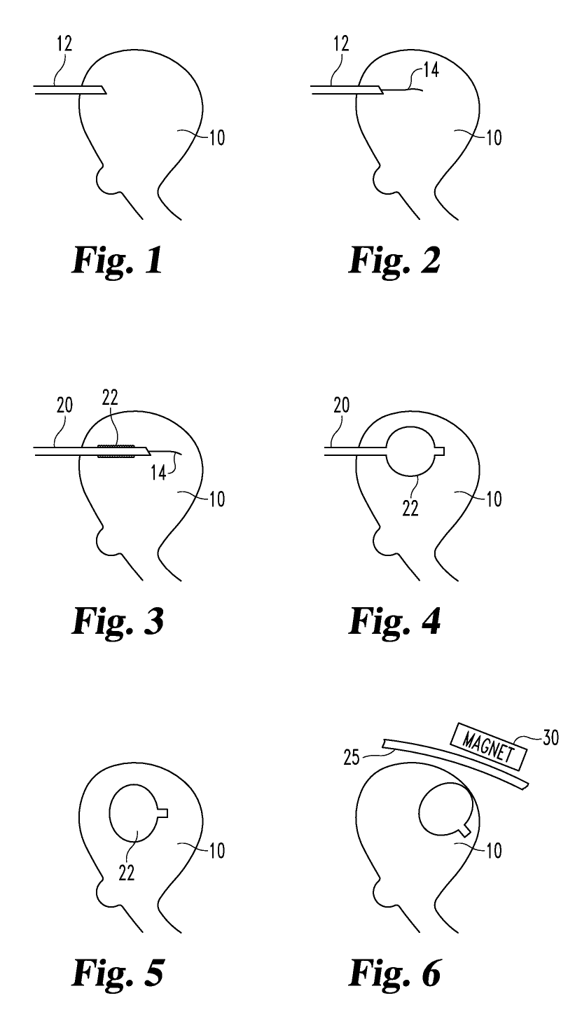

Referring now to FIGS. 1-6, use of a balloon filled with magnetic material to retract a gall bladder during a natural orifice cholecystectomy is depicted. Using a transoral approach, a hole in the stomach wall (not shown) is created to establish an access path to the peritoneal cavity. A hollow needle 12 is inserted through this path and into the gall bladder 10 to position a guide wire 14 in the gall bladder 10. A catheter 20 having a deflated balloon 22 near its distal end is then passed over the guidewire 14 and positioned with the deflated balloon 22 inside the gall bladder 10. The guidewire 14 may then be removed.

Once in position in the gall bladder 10, the balloon 22 is filled with a magnetic fluid, for example an FeSO₄ solution. When fully filled with the magnetic fluid, the balloon 22 is tied off (to prevent the magnetic fluid from being released) and detached from the delivery catheter 20. The catheter 20 may then be withdrawn, leaving the balloon 22 now filled with magnetic material in the gall bladder 10. An external magnet 30, for example a permanent magnet or electromagnet, is then positioned adjacent the skin 25 and relative to the gall bladder to attract (or repel) the balloon 22 and thereby retract the gall bladder.

With the gall bladder retracted, cutting instruments (not shown) are inserted through the surgical access path in the stomach wall and used, under endoscopic visualization, to excise the gall bladder. The resulting wound is cauterized or sutured as necessary, and removal instrument(s) (not shown) (e.g. forceps or a suction device) are inserted to grasp the now excised gall bladder, still containing the magnetic filled balloon, and withdraw it from the body via the access path. Until grasped with the removal instrument, the excised gall bladder can be maintained in position via the external magnet. Additionally, because it is effectively magnetized, the removal instruments can incorporate a magnet to help grasp onto and hold the gall bladder during removal.

FIG. 7 depicts a side view of an articulating arm 35 holding the external magnet 30. The arm 35 may be attached to a clamp 36 that clamps to the side rail 40 of the patient's bed 42, or the arm 35 and magnet 30 may be positioned in any other convenient location in the operating room. Alternatively, the external magnet 30 may be held in position by operating room personnel or attached to the patient's body (e.g. by tape).

In place of or in addition to a balloon filled with magnetic fluid, other mechanisms for delivering a sufficient quantity of magnetic material into the gall bladder so as to make it magnetically moveable with the external magnet may be employed. For example, a series of beads (made of magnetic material) can be inserted into the gall bladder. These beads can be inserted directly into the gall bladder or they may be inserted into a balloon or similar container placed in the gall bladder. In another variation, a magnetic wire can be coiled in the gall bladder.

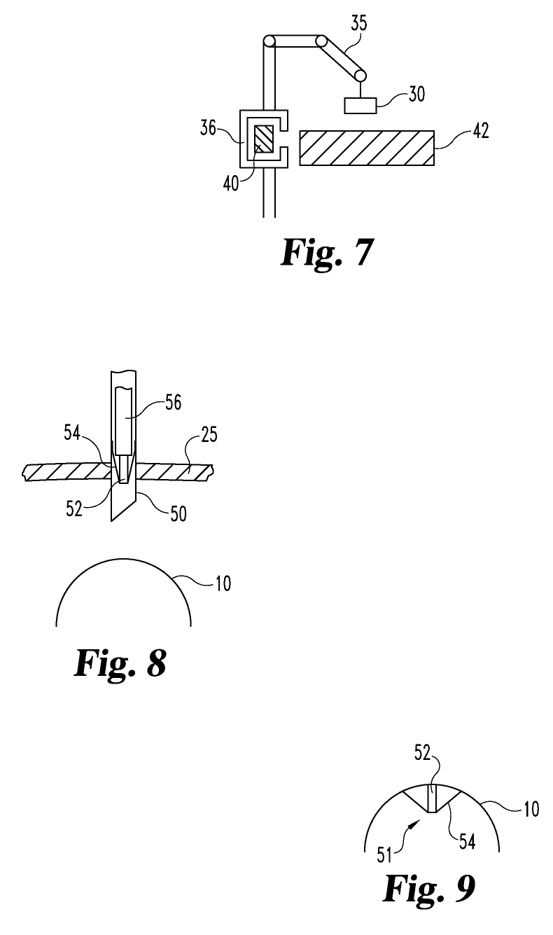

In another variation, an expandable device comprised at least partially of magnetic material can be inserted into the gall bladder. FIGS. 8 and 9 depict the insertion of one such expandable device 51 through a hollow needle 50. The device 51 has a body 52 and a number of arms 54 that are expandable relative to the body 52 and to each other. The arms 54 are collapsed when the device 51 is in the lumen of the injection needle 50. A pusher rod 56 pushes the device out the needle 50 and into the gall bladder 10. When outside the needle 50 and in the gall bladder 10, the arms 54 expand, thereby increasing the surface area over which the device 51 will contact the wall of the gall bladder when the external magnet creates the magnetic field. The device 51 may be constructed such that the arms 54 expanded by spring action (i.e. the arms are compressed by the walls of the needle and expand when the compression force is released). The arms 54 may be made of a material having super elastic properties (e.g. Nitinol) to allow a broad range of motion between the compressed (in the needle) and expanded (in the organ) states. Alternatively or in addition, the arms may be made of shape memory material (e.g. Nitinol) such that they transition from their collapsed to expanded configuration via a temperature change, for example caused by exposure to the higher temperature of the body or by application of an electric potential to the device 51.

As shown in FIGS. 8 and 9, the hollow needle 50 that serves as the delivery mechanism for the magnetic material (in this case an expandable implant) is inserted through the skin 25. This is but one useful path for accessing the organ of interest (in this case the gall bladder 10). It is to be understood that the delivery mechanism for the magnetic material/implant can be provided at the organ of interest in a variety of ways, for example transorally or transnasally (via an incision in the stomach wall).

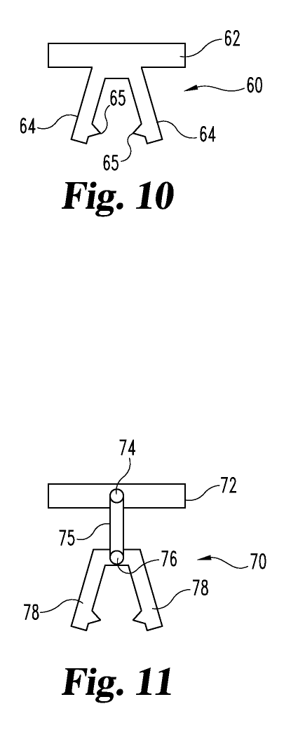

FIGS. 10 and 11 depict devices 60, 70 that can be clamped onto the gall bladder. Device 60 has a body 62 that may be made of a magnetic material. A pair of clamping components or jaws 64 (shown in the open position in FIG. 10) with tissue gripping teeth 65 extend from the body and are configured to clamp onto gall bladder. Device 70 is similar to device 60 save that the body 72 is connected to the clamping jaws 77 by an arm 75. The arm 75 is joined to the body 72 and the clamping jaws 77 via hinges 74, 76, which allow additional degree of motion between the magnetic body 72 and jaws 78 that are clamped to the gall bladder.

In devices 60, 70, the jaws 64, 78 may be constructed of shape memory materials (e.g. Nitinol) and activated between their open (as illustrated) and closed (clamped) positions by application of heat, for example heat generated by an external source (e.g. application of electric potential) or the natural body heat of the patient. In other words, at room temperature, the clamping jaws are open and when the jaws are warmed, they clamp shut.

Alternative designs are also contemplated wherein a magnetic structure is attached to the gall bladder (or other organ to be manipulated) by sutures, staples, or clips. Also, multiple magnetic devices can be attached to the gall bladder or a combination of devices/materials inserted into and attached to the gall bladder may be used.

It is to be understood that the present techniques can advantageously be used in the removal of selected organs, such as the gall bladder. In such a procedure, the magnetic material is inserted into the organ in any of the forms described above and used to retract the organ. The organ is then cut and the organ and the magnetic material are removed together from the body. Conventional techniques for grasping the excised organ can be employed, such as suctioning or clamping of the organ may be employed. Additionally, because the organ is effectively made magnetic, the organ could also be grasped for removal via a magnetic device used in place of or in conjunction with conventional forceps or suctioning devices.

The magnetic material is preferably constructed such that is can safely remain in the body. However, during organ removal, it is expected that the magnetic material only needs to remain in the organ long enough to assist in the organ removal. In other words, the magnetic material is removed with the organ and then discarded. Furthermore, because the magnetic material is contained in the organ or securely clamped to the organ, the chances of the material migrating into other parts of the body are greatly reduced.

It is to be appreciated that what has been described is a novel technique for manipulating an organ during minimally invasive surgery comprising surgically inserting a magnetic material into or onto the organ and then manipulating the organ with an external magnet. An access path into or adjacent the organ can first be established with a delivery mechanism, such as a hollow needle, a catheter or similar device having an internal lumen. The magnetic material can then be injected through the lumen of the delivery mechanism.

The magnetic material can take the form of a number of beads injected into the organ, a balloon inserted into the organ and then filled with a liquid (or with beads), a wire coiled in the organ, an expandable structure inserted in a collapsed configuration and then expanded when inside the organ, or a device that is attached (i.e. clamped, stitched, stapled) onto the organ. The magnetic material placed into or on the organ can be passively magnetic and manipulated by an active magnet external to the body. The external magnet can be mounted on an arm that is articulable relative the patient to allow the organ to be manipulated and retained in a desired position during surgery.

The delivery mechanism for the magnetic material can be positioned at or in the organ in any conventional fashion, for example endoscopically or laparoscopically via an incision in the skin. In a preferred form, one or more of the positioning of the magnetic material delivery mechanism or the removal of the organ is performed via a natural orifice approach. In other words, either the delivery mechanism or the removal of the organ (after excision), is through an incision in the stomach wall that is accessed via the patient's esophagus (e.g. transorally or transnasally) or other natural orifice (i.e. transanally). The magnetic material may be delivered and/or the delivery mechanism may be positioned under endoscopic or fluoroscopic visualization.

A useful procedure according to the present invention comprises providing a delivery mechanism defining at least one lumen, positioning the delivery mechanism at an organ of interest, and delivering magnetic material to the organ via the lumen of the delivery mechanism to thereby allow the organ to be manipulated relative to other body organs by an external magnet. For an organ removal, the organ, thusly magnetized, will be retracted by an external magnet. The delivery mechanism for the magnetic material is removed, and a cutting instrument is inserted to excise the organ. Once excised, the cutting instrument is removed and a removal device (for example having a clamp, suction, or magnet attachment member) is inserted to grasp the excised organ and withdraw it from the body. Preferably, one or all of the insertion of the deliver mechanism, the insertion of the cutting instrument(s), and the insertion of the organ removal device occurs through a common pathway. In a further preferred form, this common pathway is through an incision in the stomach wall (e.g. via a NOTES type procedure).

What is claimed is:

1. A method for manipulating an internal organ of a patient during a minimally invasive procedure comprising:
delivering magnetic material into or onto the internal organ; and
manipulating the organ during the procedure with a magnet external to the patient's body.

2. The method of claim 1 wherein the organ is removed during the procedure.

3. The method of claim 2 wherein the organ and the magnetic material are removed from the patient together.

4. The method of claim 1 wherein delivering the magnetic material includes:
inserting a flexible membrane into the organ; and
filling the membrane with a fluid comprising magnetic material.

5. The method of claim 1 wherein delivering the magnetic material includes injecting a number of bead into the organ.

6. The method of claim 1 wherein delivering the magnetic material includes coiling a wire inside the organ.

7. The method of claim 1 wherein delivering the magnetic material includes inserting a self expanding structure having a magnetic component into the organ.

8. The method of claim 7 wherein the self expanding structure expands when pushed out the lumen of a delivery device.

9. The method of claim 1 wherein delivering the magnetic material includes clamping a device onto the organ.

10. The method of claim 1 wherein the magnetic material is delivered to the organ via an access path including a natural body orifice.

11. The method of claim 10 wherein the access path is established through a wall of the stomach.

12. A method of operating on an organ in the peritoneal cavity of a patient comprising:
establishing an access path to the organ in the peritoneal cavity through a hole cut in the wall of the stomach;
using the access path to deliver magnetic material into or onto the organ; and
manipulating the organ with a magnet external to the patient's body.

13. The method of claim 12 further comprising:
removing the organ and the magnetic material via the access path.

14. A surgical system comprising:
an elongated body adapted to be inserted through the esophagus and into the peritoneal cavity through a hole cut in the wall of the stomach, the body including a delivery lumen;
magnetic material adapted to be delivered through the delivery lumen of the elongated body and into or onto an organ in the peritoneal cavity; and
a magnet external to the patient for manipulating the organ once it has been magnetized with the magnetic material.

15. The surgical system of claim 14 wherein the magnet is mounted on an articulating arm.

16. The surgical system of claim 14 wherein the magnetic material includes a number of beads.

17. The surgical system of claim 14 wherein the magnetic material is fluidized.

18. The surgical system of claim 14 wherein the magnetic material is a part of a self expanding member.

19. The surgical system of claim 14 wherein the magnetic material is provided on a device configured to be clamped onto the organ.

20. The surgical system of claim 14 wherein the magnetic material includes a wire.
